(19) United States
(12) Patent Application Publication
(10) Pub. No.: US 2016/0081858 A1
(43) Pub. Date: Mar. 24, 2016

(54) Compositions and Methods for the Prevention of Scarring and/or Promotion of Wound Healing

(71) Applicant: DUKE UNIVERSITY, Durham, NC (US)

(72) Inventors: Howard Levinson, Durham, NC (US); Kam W. Leong, Durham, NC (US); Elizabeth R. Lorden, Durham, NC (US)

(73) Assignee: DUKE UNIVERSITY, Durham, NC (US)

(21) Appl. No.: 14/785,264

(22) PCT Filed: Apr. 16, 2014
(86) PCT No.: PCT/US14/34378

§ 371 Date: Oct. 16, 2015

Related U.S. Application Data
(60) Provisional application No. 61/812,312, filed on Apr. 16, 2013.

(51) Int. Cl. A61F 13/00; A61F 2/12

(52) U.S. Cl. CPC A61F 13/00063; A61F 2/12; A61F 13/00017; A61F 2013/00357

(57) ABSTRACT

The present disclosure provides methods of preventing and/or reducing scar contracture and methods of promoting wound healing by utilizing an electrospun biocompatible scaffold.

### CROSS REFERENCE TO RELATED APPLICATIONS

This application claims the benefit of priority to U.S. Provisional Patent Application Ser. No. 61/812,312 filed Apr. 16, 2013, which is incorporated herein by reference in its entirety.

### FEDERAL FUNDING LEGEND

This invention was produced in part using funds from the Federal Government under NIH Grant Nos: EB01.5000, UH2TR00050.5, K08 GM085562. Accordingly, the Federal Government has certain rights to this invention.

### BACKGROUND

Chronic non-healing wounds and pathologic scars are major medical problems that are estimated to cost billions of dollars annually. (Langer, A. & Rogowski, (2009)). In the United States, chronic wounds affect 6.5 million patients and cost approximately $25 billion annually to treat. The annual wound care products market alone is projected to reach $15.3 billion by 2010. (Sen, C. K. et al. (2009)).

There are approximately 34 million American patients who undergo surgical procedures. Two million people are injured in motor vehicle accidents and over 2.4 million patients are burned. In severe burns and blast injuries, more than 40% of patients develop large joint scar contractures. (Schneider, J. C., et al, (2006)). In terms of breast reconstruction and augmentation, it is estimated that 8.08 per 1.000 women in the United States, or approximately 815,000 women reported having had some type of breast implant.

Between both chronic wounds and scars, there are more than 40 million Americans affected by these medical problems annually. Scars are a major concern in and reconstructive procedures as there are currently no effective anti-scarring devices or

The present disclosure addresses these shortcomings by utilizing novel bioengineered scaffolds, and methods of using said scaffolds, to prevent scarring and promote wound healing.

### SUMMARY OF THE INVENTION

The present disclosure provides compositions and methods of promoting wound healing and preventing and/or reducing scar contractures using electrospun biocompatible scaffolds. Such scaffolds are described in detail in US Patent Publication Nos, 2010/0055154, 2001/0142806, and 2012/0141547, the contents of which are hereby incorporated by reference in their entirely.

One aspect of the present disclosure provides a method of promoting wound healing in a subject comprising, consisting of, or consisting essentially of implanting an electrospun biocompatible scaffold in the wound of the subject to promote granulation tissue formation and to facilitate epithelialization.

Another aspect of the present disclosure provides a method of preventing and/or reducing scar contracture in a subject comprising, consisting of, or consisting essentially of implanting an electrospun biocompatible scaffold in the wound of a subject to minimize mechanical strain transmission and/or reduce cellular contraction thereby preventing and/or reducing scar contracture.

Another aspect of the present disclosure provides a method of preventing reducing capsular contractures in breast reconstruction and/or augmentation procedures comprising, consisting of, or consisting essentially of (a) wrapping an electrospun biocompatible scaffold around a breast implant; and (b) implanting the breast implant into the subject, the scaffold thereby minimizing mechanical strain transmission and/or reducing inflammation thereby preventing and/or reducing capsular contractures.

In one embodiment, the scaffold in implanted subcutaneously.

In one embodiment, the wound comprises a chronic wound. In some embodiments, the chronic wound comprises a venous stasis ulcer. In another embodiment, the chronic wound comprises a diabetic foot ulcer.

In another embodiment, the electrospun biocompatible scaffold comprises polyurethane (PU).

The methods and biocompatible scaffolds described herein can be used in trauma, cancer, and infection reconstruction. Trauma, cancer, and infections all cause large wounds. Large wounds heal by forming a granulation bed, predominantly consisting of collagen and also containing active fibroblasts, immune cells, and blood vessels. The granulation bed is skin grafted or a provisional collagen scaffold is placed, granulation tissue forms within the interstices of the scaffold and the scaffold is skin grafted 2-3 weeks after placement. Beneath the skin graft, the granulation bed continues to mature for up to 6 months. In the maturation phase, fibroblasts differentiate into contractile myofibroblasts which serve to contract and stiffen the extracellular matrix (ECM). (Schneider, J. C. et al. (2006)). At the cessation of healing, these cells typically undergo a massive wave of apoptosis however in patients with scar contracture this does not occur. Rather, the myofibroblasts persist in the wound bed where they continue to contract the ECM and activate surrounding fibroblasts to differentiate into the contractile myofibroblast phenotype. The pathologic ECM contraction caused by this positive feedback loop leads to scar contracture. Contractures are fixed deformities that are aesthetically displeasing, painful, itchy, and functionally debilitating. Contractures are a direct response to mechanical strain and soluble substances, such as those produced by inflammatory mediators, in the wound.

Collagen scaffolds in use today were developed to expedite healing of wounds; they were not developed based on the knowledge of biological mechanisms leading to scar contractures. While recent studies have evaluated the potential anti-scarring properties of collagen scaffolds, these scaffolds have several undesirable characteristics that permit mechanical strain transmission and exacerbate inflammation. (Doshi, J. & Reneker, D. H. (1995)). Moreover, they are expensive to manufacture, concerns for disease transmission linger, and efficacy is influenced by patient-to-patient variability. (Taylor, G. (1969)). For these reasons, start-of-the-art collagen based scaffolds are unlikely to ever achieve the goal of preventing scar contracture.

Therefore, biocompatible scaffolds described herein have the potential to promote regeneration while minimizing scar contracture. Viscoelastic biocompatible materials, such as Polyurethane (PU), have material properties that minimize mechanical strain transmission. Therefore, in accordance with one embodiment of the present disclosure, a unique PU scaffold with appropriate mechanical properties for use in dermal tissue regeneration has been developed.

Yet another aspect of the present disclosure provides for all that is disclosed and illustrated herein.

### BRIEF DESCRIPTION OF THE DRAWINGS

The foregoing aspects and other features of the invention are explained in the following description, taken in connection with the accompanying drawings, wherein:

FIG. 1 is a schematic detailing the method of making a biocompatible scaffold according to one embodiment of the present disclosure.

FIG. 2 are photographs of PU Scaffolds with fiber diameter of 4-6 μm (A) and total thickness of 100 μm (B).

FIG. 3 is a representation of the physical and mechanical properties of PU scaffolds. (A) Fatigue studies show maintenance of elasticity under physiologically relevant conditions (15,000 cycles at 1 Hz in 37° C. Phosphate Buffered Saline solution), indicating that PU will not lose its elasticity due to repetitive joint motion. Each line of A represents a single sample: small variations around cycle 1000 are due to additional fluid being added to the sample cup. (B) PU scaffolds under environmental scanning electron microscopy (ESEM) maintain their topography under (C) 100% strain, indicating that elongation during joint motion will not disrupt scaffold architecture. (D) Stress-strain data from static tensile tests show that PU is tougher than Integra™ (Integra Lifesciences, New Jersey), and thus, would rupture with repeated joint motion, (E) Elastic modulus of PU is less than or equal to unwounded human skin, indicating that PU will never impede joint motion. (F) Ultimate tensile stress is greater than or equal to human skin, indicating that the scaffold will not tear under extreme stress placed across the skin, and (G) Elongation at break is greater than or to that of human skin, indicating that PU will not break under physiologic stresses and strains. Together, D-G show that NJ is mechanically appropriate for application as a bioengineered skin equivalent (BSE), and has superior mechanical properties to Integra™.

FIG. 4 illustrates that scaffolds prevent scar contracture related markers in vitro compared with fibroblast populated collagen lattice (FPCL) (A) Contraction studies were performed on FPCLs and PU scaffolds; black dashed lines outline PU and FPCLs at each time point to show changes in area, (B) FPCLs contracted rapidly while PU scaffolds retained their original area over seven days in culture; analyses of changes in area were performed by computer planimetry. (C) On day 7, cells were fixed and stained for αSMA and DAPI. Significantly more αSMA positive cells were found in FPCLs than in PU scaffolds. Images were analyzed using Image J software.

FIG. 5 demonstrates PU scaffolds inhibiting scar contraction and displaying minimal immune reaction at 21 days. Skin grafts were placed on C57BL/6 mice (n=8). Controls received skin graft alone. Test groups were skin grafts with PU scaffolds (110 μm thick), or standard of care Integra™. (A) Representative photographs of mice contraction of wounds. (B) Scar contraction was nearly totally inhibited by PU scaffolds, as compared to Integra™ and controls. (C) Quantitative analysis and representative images of macrophages (black arrows show macrophages stained in brown) in the wound bed show similar immune responses between Integra™ and PU Scaffolds on day 14. The average of 5 HPFs of 3 samples were graphed for figure C. Statistical significance evaluated by the of variance (ANOVA), followed by least significant difference t-test. All values used in and text are expressed as mean±SEM, p-value<0.05.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless specifically defined, all technical and scientific terms used herein have the same meaning as commonly understood by a skilled artisan in engineering, biochemistry, cellular biology, molecular biology, cosmetics, and the medical sciences (e.g., dermatology, etc.). All methods and materials similar or equivalent to those described herein can be used in the practice or testing of the present disclosure, with suitable methods and materials being described herein.

Articles “a” and “an” are used herein to refer to one or to more than one (i.e. at least one) of the grammatical object of the article. By way of example, “an element” means at least one element and can include more than one element.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of elements that do not materially affect the basic and novel or functional characteristic(s) of that embodiment of the invention.

The term “consisting of” refers to methods and respective components thereof as described herein, which are exclusive of any element not recited in that description of the embodiment.

As used herein, the term “subject” is intended to include human and non-human animals. Exemplary human subjects include a human patient having a disorder, e.g., a disorder described herein, or a normal subject.

As used herein, a “scaffold” may comprise any biocompatible material that is capable of being electrospun. Examples include, but are not limited to polyurethane (PU), poly(caprolactone), poly(ethylene oxide), CP2, PVDF, poly(dimethylsiloxan) (PDMS), polystyrene, poly L-lactic acid, poly glycolic acid, poly hydroxybutyrate, polycarbonate (PC), polycaprolactone (PCL), polymethylmethacrylate (PMMA), or other thermoplastic polymers or combinations thereof. In certain embodiments, the scaffold comprises Polyurethane because of its unique elastomeric properties. For example, when PU is placed across a joint the mesh could expand and contract without plastic deformation. (Britannica).

As used herein, the term “chronic wound” refers to those wounds that do not heal in an orderly set of stages and in a predictable amount of time. Typically, wounds that do not heal within three months are considered chronic. Causes of chronic wounds are numerous, and may include poor circulation, age, neuropathy, difficulty in moving, systemic illnesses, repeated trauma, inflammation, immune suppression, pyoderma gangrenosum and diseases that cause ischemia. Examples of chronic wounds include, but are not limited to, venous stasis ulcers, diabetic foot ulcers, and the like. For instance, for the treatment of diabetes and venous stasis ulcers, the scaffold is applied to the wounds to promote granulation tissue formation and facilitate epithelialization. Chronic, wounds may also include those relating to trauma (or repeated trauma), thermal injury (e.g., burns) and radiation damage.

As used herein, the term “prevention” means generally the prevention, reduction, or mitigation of the establishment of scar formation, scar contracture, or capsular contractures in a subject that may or may not have exhibited a need for scar formation.

The following examples are offered by way of illustration and not by way of limitation.

### EXAMPLES

### Example 1

### Electrospun Biocompatible Scaffold Fabrication

Electrospinning as a Tunable Technique for Biomaterial Fabrication.

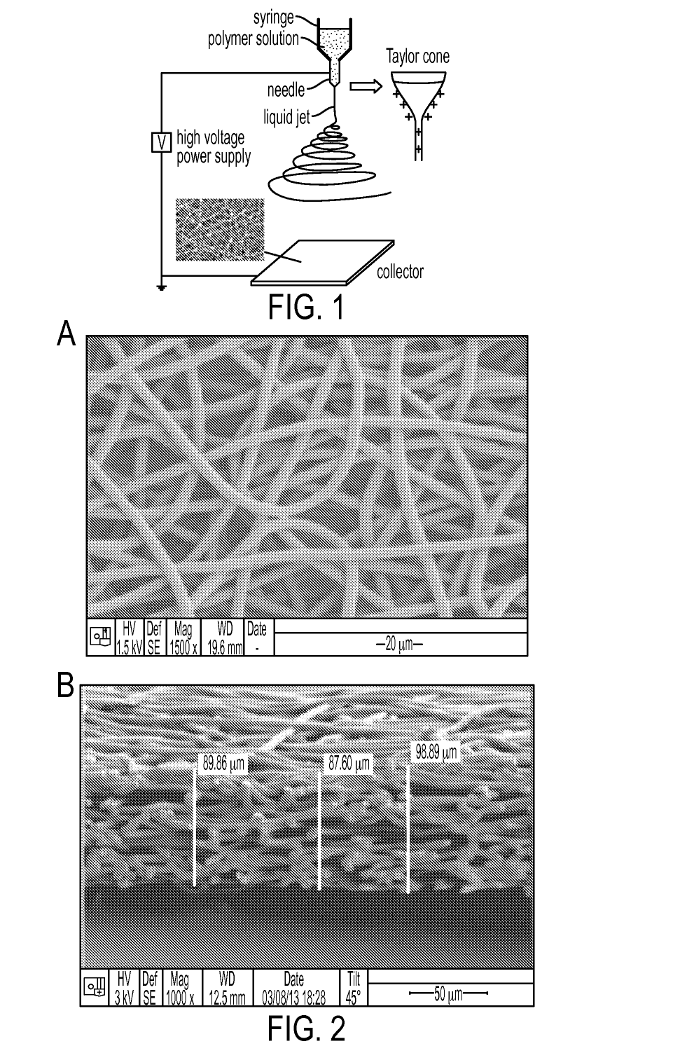

Over the past two decades, electrospinning has become a fabrication technique for tissue engineering due to its simplicity and versatility to fine-tune the mechanical and mass transport properties of the scaffolds. The fibrous nature also provides topographical cues to adherent cells. As shown in FIG. 1, electrospinning can be carried out by dissolving a polymer in an organic solvent, placing the solution inside a syringe and ejecting the solution through a charged needle at a constant rate. When placed in an electrical field, the electrical force overcomes the viscous three of the polymer solution droplet hanging on the needle to create a spinning jet towards a grounded surface (Doshi and Reneker; Gururajan et al., (2011); Taylor ((1969)). In flight, the solvent evaporates and polymer fibers with diameters in the nano-micrometer range are deposited on the collecting surface.

Fiber characteristics are tailored by changing polymer type, molecular weight, concentration, solvent evaporation rate, applied voltage, solution flow rate, ambient temperature and humidity, and distance from needle to ground. (Chakraborty et al., (2009)). Fiber alignment is controlled by changing the above parameters as well as the speed at which the collecting mandrel rotates or including extra hardware, such as a ring electrode setup, for example. Changes in fiber alignment directly impact the macroscopic mechanical properties the electrospun matrix. (Baji et at (2010)). Our studies have shown that the physical and mechanical properties of PU meshes can be optimized by using different molecular weights Cardioflex Polyurethane and controlling the electrospinning fabrication parameters. (Liao et (2008)). Electrospinning can produce fibers mimicking the nanotopographical features in the extracellular matrix of tissues. (Murugan and Ramakrishna, (2007)). Electrospun fibrous scaffolds can be applied towards a broad range of regenerative medicine applications, including dermal wound healing. (Choi et al., (2008)).

The scaffold may comprise any biocompatible material that is capable of being electrospun. Examples include, but are not limited to, polyurethane (PIT), poly(caprolactone), poly(ethylene oxide), CP2, PVDF, poly(dimethylsiloxan) (PDMS), polystyrene, poly L-lactic acid, poly glycolic acid, poly hydroxybutyrate, polycarbonate (PC), polycaprolactone (PCL), polymethylmethacrylate (PMMA), or other thermoplastic polymers or combinations thereof, in certain embodiments, the scaffold comprises Polyurethane because of its unique elastomeric properties. For example, when PU is placed across a joint the mesh could expand and contract without plastic deformation. (Britannica).

Electrospinning:

As previously described, the scaffold according to the present disclosure are created by electrospinning. Electrospinning is a technology which utilizes electrical charge to overcome the surface tension of a polymer solution in order to shear the polymer solution into micro-to-nanoscale fibers. Fibers having diameters that are less than one micron are often referred to as “nanofibers”. Fibers having diameters equal to or greater than one micron are often referred to as microfibers. The electrospinning can be adjusted to modify topography of the scaffold, including but not limited to surface area:volume, porosity, and fiber alignment.

The scaffold (e.g., NJ scaffold) essentially serves as a biomimetic neomatrix that enables new tissue ingrowth and facilitates tissue regeneration. Biocompatible and biostable implants have the advantage that they will not release any degradation byproducts that may inhibit the healing process or cause local/systemic toxicity. When a polyester electrospun mesh is embedded it is shown to intimately associate with new dermal tissue and not compromise neodermis formation. (Cahn and Kyriakides, (2008)). Other biostable implants such as non-degradable sutures (Ethicon) and polypropylene mesh used for hernia repair. (Mottin et al., (2011)) can be utilized. Medical grade PU (Cardioflex AL80A) for this project has been shown to be biocompatible in our studies. (Liao and Leong, (2011); Liao et al., (2008)). When implanted subcutaneously the PU electrospun membrane showed cellular infiltration but minimal macrophage recruitment at one week, followed by integration of the fibrous membrane with nearly seamless interface into the host tissue at one month and macrophage retreat. (Liao and Leong, (2011)). This technology has potential uses tissue regeneration, including but not limited to expediting wound healing in chronic wounds caused by diabetes, venous stasis ulcers, trauma, thermal injury or radiation damage, and/or reduction of scanting following trauma, cancer reconstruction, infections, following insertion of prosthetic breast implants, or other aesthetic procedures.

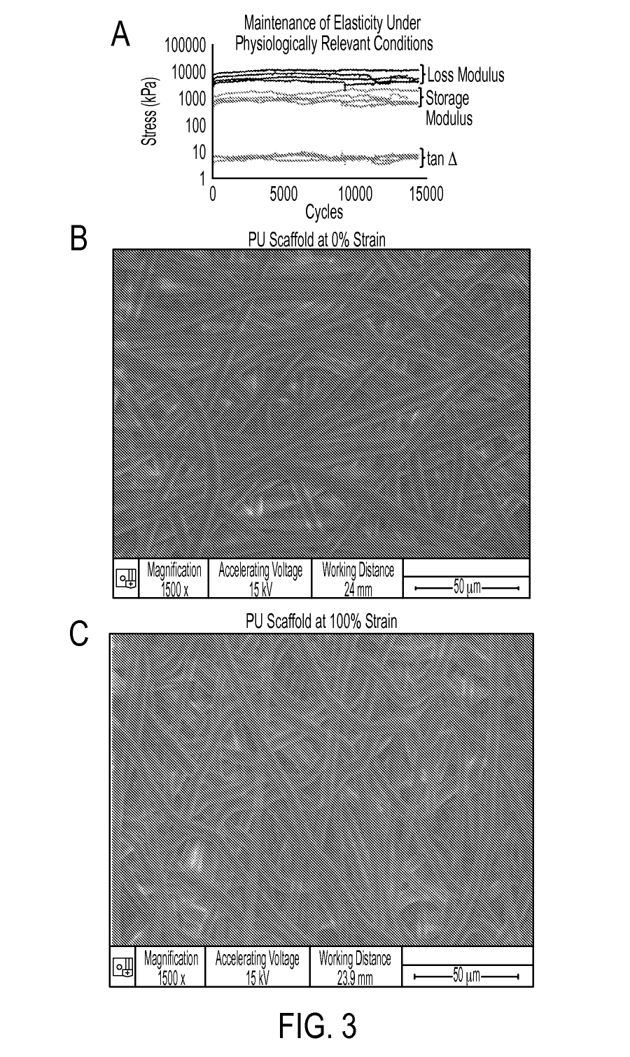

In one embodiment, the scaffolds will be fabricated by continuous single fiber electrospinning to deposit a 3D matrix of fibers on a rotating grounded mandrel. Following spinning, fibers will be removed from the mandrel and any remaining organic solvent will be allowed to fully evaporate by placing the fibers in vacuum overnight. Fiber characteristics can be tailored by changing polymer type, molecular weight (MW), solvents, concentration, applied voltage, solution flow rate, ambient temperature and humidity, and distance from needle to mandrel. In the presented work, we began with polymer type and MW as a constant (PU Cardioflex AL80A, Cardiotech International Inc.) and varied the remaining parameters to obtain uniform PU scaffolds for physical and mechanical testing. Once the electrospinning parameters were optimized, time of spinning was used to vary the scaffold thickness (50-600 μm), fiber diameter, and fiber alignment. See FIG. 2 for example of PU scaffolds.

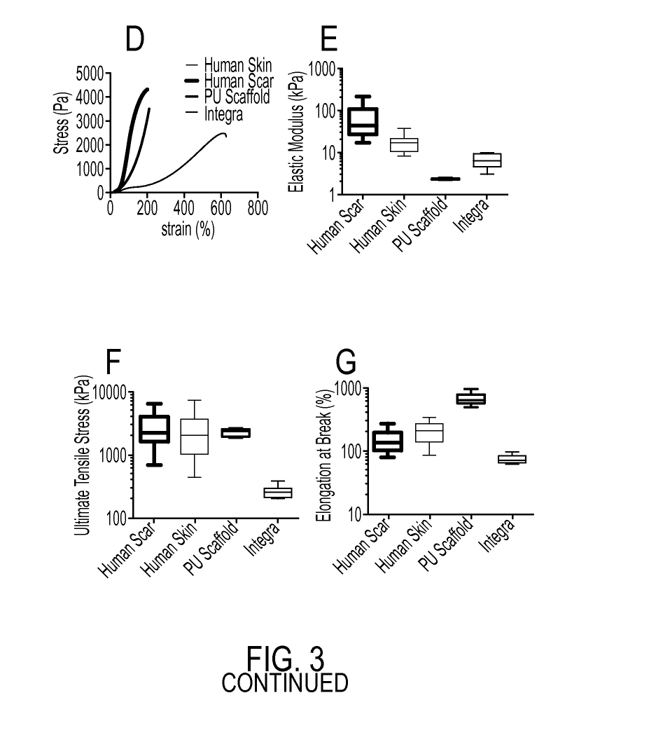

PU scaffolds were fabricated using the above methods with a random pattern topography, controlled fiber diameter (3-7 μm diameter), and heterogeneous pore size (5-60 μm). Electrospinning was selected as a fabrication method because it generates fibers mimicking the micro- and nano-topographical features in the extracellular matrix of tissues. We compared our PU scaffolds, against standard of care bioengineered skin equivalent or (Integra™), human skin tissue, and human scar tissue, for five key mechanical characteristics: maintenance of elasticity at physiological conditions (FIG. 3A.); maintenance of topography under strain (FIG. 3B, C.); and tensile stress-strain characteristics including (FIG. 3D), the elastic modulus (FIG. 3E), ultimate tensile strength (FIG. 3F), and elongation at break (FIG. 3G). Together these data show that PU scaffolds have appropriate mechanical for implantation beneath skin graft in the wound bed, and have superior mechanical to Integra™ for applications as a BSE.

### Example 2

### Methods of Using Electrospun Biocompatible Scaffolds

Promoting Wound Healing:

One aspect of the present disclosure provides a method of promoting wound healing in a subject comprising, consisting of, or consisting essentially of implanting an electrospun biocompatible scaffold in the wound of the subject to promote granulation tissue formation and to facilitate epithelialization. In some embodiments, the wound comprises a chronic wound. Typically, wounds that do not heal within three months are considered chronic. Causes of chronic wounds are numerous, and may include poor circulation, age, neuropathy, difficulty in moving, systemic illnesses, repeated trauma, inflammation, immune suppression, pyoderma gangrenosum and diseases that cause ischemia. Examples of chronic wounds include, but are not limited to, venous stasis ulcers, diabetic foot ulcers, and the like. For instance, for the treatment of diabetes and venous Stasis Ulcers, the scaffold is applied to the wounds to promote granulation tissue formation and facilitate epithelialization.

Chronic wounds may also include those relating to trauma (or repeated thermal injury (e.g., burns) and radiation damage. For example, difficult to heal wounds are often managed by Integra™ (Integra Life Sciences, Plainsboro, N.J.) (http://www.integralife.com/products.aspx#Oti). Integra is a two-layer skin regeneration system. The outer layer is made of a thin silicone film and the inner layer is constructed of a complex matrix of resorbable cross-linked fibers. The porous material acts as a scaffold for regenerating dermal skin cells, which enables the re-growth of a functional dermal layer of Once dermal skin has regenerated, typically 2-3 weeks after Integra™ placement, the silicone outer layer is removed and replaced with a thin epidermal skin graft. One main advantage of biocompatible scaffolds, and methods provided herein, over Integra™ in the chronic wound space is that there is no 2-3 week waiting period for skin graft application.

Preventing/Reducing Scar Contracture:

Another aspect of the present disclosure provides a method of preventing and/or reducing scar contracture in a subject comprising, consisting of, or consisting essentially of implanting an electrospun biocompatible scaffold in the wound of a subject to minimize mechanical strain transmission thereby preventing and/or reducing scar contracture. Scars are a major concern in aesthetic and reconstructive procedures. There are currently no effective anti-scarring devices or drugs.

Biocompatible scaffolds described herein have the potential to promote regeneration while minimizing scar contracture. Viscoelastic biocompatible materials, such as Polyurethane (PU), have material properties that minimize mechanical strain transmission. Therefore, in accordance with one embodiment of the present disclosure, a unique PU scaffold with appropriate mechanical properties for use in dermal tissue regeneration has been developed.

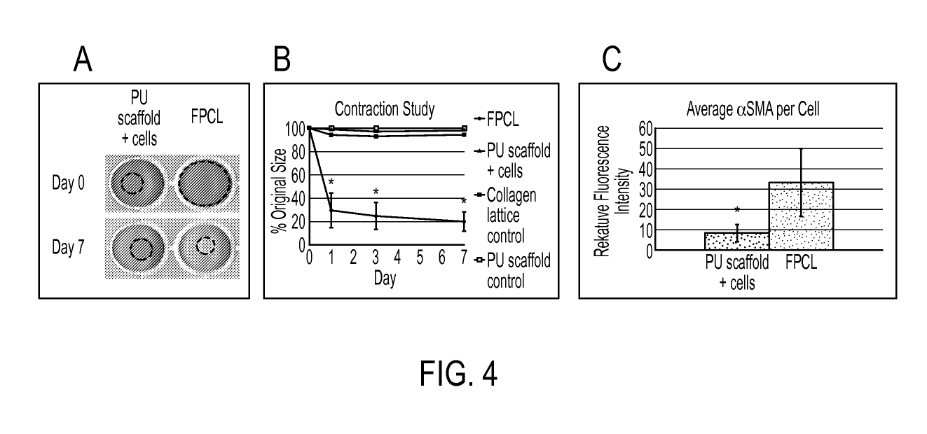

Mechanistically, a viscoelastic scaffold would absorb mechanical tension without transmitting forces to the fibroblast and would thus reduce fibroblast-to-myofibroblast differentiation through downstream mechanisms. Data demonstrate that when compared to the fibroblast populated collagen lattice (FPCL) wound contraction model. PU scaffolds prevent matrix contraction (FIG. 4A, B) and fibroblast-to-myofibroblast differentiation (as shown by alpha smooth muscle actin (αSMA) staining in FIG. 4C)).

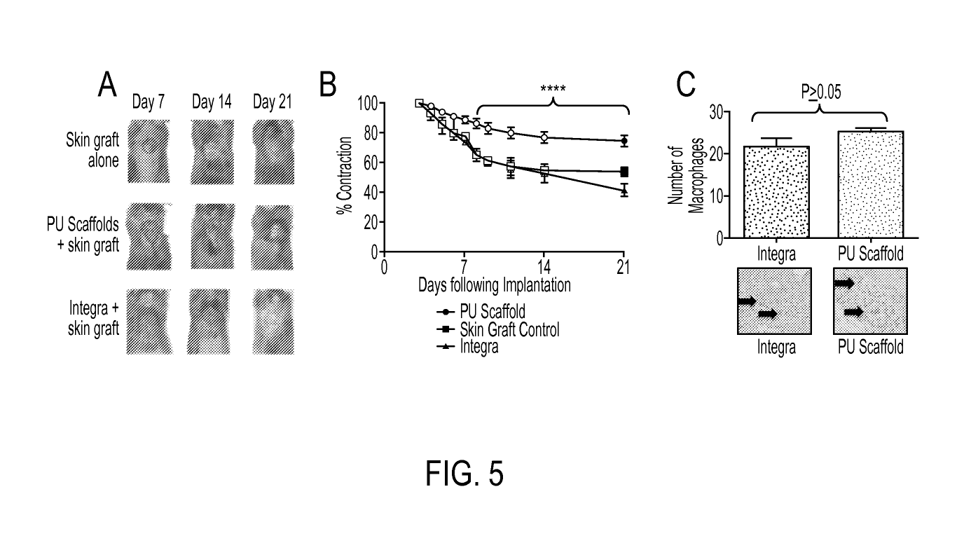

Electrospun PU scaffolds, Integra™, and skin grafts alone were tested in immune-competent murine scar contracture model to determine their effectiveness at preventing scar contraction in vivo. Performance was analyzed according to: 1) ability to prevent or minimize scar contraction (computer planimetry), and 2) ability to minimize foreign body response (F4/80 staining). Results from these studies show that, in contrast to Integra™, the PU scaffolds almost completely inhibited scar contraction through 21 days (FIG. 5A, 5B). In addition to gross observations of success, a minimal foreign body response was observed via immunohistology on day 14 (FIG. 5C).

### Example 3

### Methods of Using Electrospun Biocompatible Scaffolds for Breast Reconstruction

Breast Reconstruction and Augmentation:

Yet another aspect of the present disclosure provides a method of preventing and/or reducing capsular contractures in breast reconstruction and/or augmentation procedures comprising, consisting of, or consisting essentially of (a) wrapping an electrospun biocompatible scaffold around a breast implant; and (b) implanting the breast implant into the subject, the scaffold thereby minimizing mechanical strain transmission thereby preventing and/or reducing capsular contractures.

Prosthetic breast implants are the most common approach to reconstructing the breast after cancer or birth defects and in aesthetic augmentation. All breast implants are foreign bodies, and as such, all implants develop a capsule because of the foreign body response. Up to 15% of capsules contract. Capsular contractures are painful and disfiguring. They are a leading cause for breast implant removal and further surgery. There is currently no therapy to prevent capsular contractures. The biocompatible scaffolds and methods of using said scaffold described herein can be used in such procedures by wrapping the scaffold around breast implants to prevent capsular contracture for the same rationale why PU scaffolds prevent skin contractures. Furthermore, it is estimated that patients will pay $500-$1500 per procedure for the addition of a device that will prevent scarring. Since scarring is caused by mechanical strain, it is expected that the biocompatible scaffolds and methods described herein may be used to mitigate scaring in the aesthetic market.

In some embodiments, such as for wound healing, the biocompatible scaffolds are implanted subcutaneously. In other embodiments, such as for breast reconstruction/augmentation, the biocompatible scaffold is wrapped around the implant and placed within the body where the implant is needed. In yet other embodiments, the biocompatible scaffold is placed within a body cavity.

Any patents or publications mentioned in this specification are indicative of levels of those skilled in the art to which the invention pertains. These patents and are herein incorporated by reference to the same extent as if each individual publication was specifically and individually indicated to be incorporated by reference. In case of conflict, present specification, including definitions, will control.

One skilled in the art will readily appreciate that the present invention is well adapted to can out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### REFERENCES

1. Langer, A. & Rogowski W. Systematic review of economic evaluations of human cell-derived wound care products for the treatment of venous leg and diabetic foot ulcers. BMC Health Serv Res 9, 115 (2009).
2. Sen, C. K. et al. Human skin wounds: a major and snowballing threat to public health and the economy. Wound Repair Regen 17, 763-71 (2009).
3. Schneider, J. C., Holavanahalli, R., Helm, P., Goldstein. R. & Kowalske, K. Contractures in burn injury: defining the problem. J Burn Care Res 27, 508-14 (2006).
4. Doshi, J. &. Reneker, D. H. Electrospinning Process and Applications of Electrospun Fibers. J. Electrost. 35, 151-160 (1995).
5. Taylor, G. Electrically Driven Jets. Proceedings of the Royal Society of London Series a-Mathematical and Physical Sciences 313, 453-457 (1969).
6. Gururajan, G., Sullivan, S. F. Beebe, T. P., Chase, D. B. & Rabolt, J. F. Continuous electrospinning of polymer nanofibers of Nylon-6 using an atomic force microscope tip. Nanoscale 3, 3300-8 (2011).
7. Chakraborty, S., Liao, I. C., Adler, A. & Leong, K. W. Electrohydrodynamics: A facile technique to fabricate drug delivery systems. Adv Drug Deliv Rev 61, 1043-54 (2009).
8. Baji, A., Mai, Y.-W., Wong, S.-C., Abtahi. M. & Chen. P. Electrospinning of polymer nanofibers: Effects on oriented morphology, structures and tensile properties. Composites Science and Technology 70, 703-718 (2010).
9. Liao, I. C., Liu, J. B. Bursac, N. & Leong, K. W. Effect of Electromechanical Stimulation on the Maturation of Myotubes on Aligned Electrospun Fibers. Cell Mol Bioeng 1, 133-145 (2008).
10. Mulligan, R. & Ramakrishna, S. Design strategies of tissue engineering scaffolds with controlled fiber orientation. Tissue Eng 13, 1845-66 (2007),
11. Choi, J. S., Leong & Yoo, H. S. In vivo wound healing of diabetic ulcers using electrospun nanofibers immobilized with human epidermal growth factor (EGF). 29, 587-96 (2008).
12. Britannica E. Polyurethane. in Encyclopedia Britannica Online Academic Edition (Encyclopedia Britannica Inc, 2012).
13. Cahn, F. & Kyriakides, T. R. Generation of an artificial skin construct containing a non-degradable fiber mesh: a potential transcutaneous interface. Biomed Mater 3, 034110 (2008),
14. Ethicon., Ethicon Product Catalog, in Sutures: Non-absorbable (2009).
15. Mottin, C. C., Ramos, R. J. & Ramos, M. J. Using the Prolene Hernia System (PHS) for inguinal hernia repair. Rev Col Bras Cir 38, 24-7 (2011).
16. Liao, I. C. & Leon K. W. Efficacy of engineered EV III-producing skeletal muscle enhanced by growth factor-releasing co-axial electrospun fibers. Biomaterials 32, 1669-77 (2011).
17. Harrison, C. A. & MacNeil, S. The mechanism of skin graft contraction: an update on current research and potential future therapies. Burns 34, 153-63 (2008).
18. Aarabi, S Longaker, M. T. & Gurtner, G. C. Hypertrophic scar formation following burns and trauma: new approaches to treatment. PLoS Med 4, e234 (2007),
19. van Zuijlen, P. P. et al. Dermal substitution in acute burns and reconstructive surgery: a subjective and objective long-term follow-up. Plast Reconstr Surg 108, 1938-46 (2001).

1. A method of promoting wound healing in a subject comprising implanting an electrospun biocompatible scaffold in the wound of the subject to promote granulation tissue formation and/or to facilitate epithelialization.

2. A method of preventing and/or reducing scar contracture in a subject comprising implanting an electrospun biocompatible scaffold in the wound of a subject.

3. A method of preventing and/or reducing capsular contractures in breast reconstruction and/or augmentation procedures comprising (a) wrapping an electrospun biocompatible scaffold around a breast implant; and (b) implanting the breast implant into the subject.

4. The method as in claims 1-3 in which the method minimizes mechanical strain transmission.

5. The method as in claims 1-3 in which the method further prevents or reduces scar contracture.

6. The method as in claims 1-3 in which the scaffold is implanted subcutaneously.

7. The method as in claims 1-3 in which the scaffold comprises polyurethane (PU).

8. The method according to claim 1, wherein the wound comprises a chronic wound.

9. The method according to claim 8, wherein the chronic wound comprises a venous stasis ulcer.

10. The method according to claim 8, wherein the chronic wound comprises a diabetic foot ulcer.

11. An electrospun biocompatible scaffold produced by continuous single fiber electrospinning of nanofibers and/or microfibers.
